(19) United States
(12) Patent Application Publication
(10) Pub. No.: US 2018/0279945 A1
(43) Pub. Date: Oct. 4, 2018

(54) VEIN VISUALIZATION DEVICE

(71) Applicant: KOHKEN MEDICAL CO., LTD., Bunkyo-ku, Tokyo (JP)

(72) Inventors: Eiichi MATSUI, Bunkyo-ku, Tokyo (JP); Yasuo NAKAJIMA, Bunkyo-ku, Tokyo (JP); Hikaru SUZUKI, Kawasaki-shi, Kanagawa (JP)

(21) Appl. No.: 15/756,339

(22) PCT Filed: Sep. 6, 2016
(86) PCT No.: PCT/JP2016/076127

§ 371 Date: Feb. 28, 2018

(30) Foreign Application Priority Data
Sep. 30, 2015 (JP) JP2015-194188

(51) Int. Cl. A61B 5/00; A61B 90/50; A61M 5/42; H04N 5/33; H04N 5/04; G06K 9/46

(52) U.S. Cl. CPC A61B 5/489; A61B 5/0075; A61B 5/742; A61B 90/50; G01B 11/24; H04N 5/33; H04N 5/04; G06K 9/46; A61M 5/427

(57) ABSTRACT

A compact, lightweight vein visualization device excellent in operability. The non-contact type vein visualization device is a non-contact type vein visualization device comprising: an irradiating unit, which irradiates a puncture site with light containing a wavelength component of 900 to 1500 nm, an image capturing unit, which includes an infrared transmission filter and receives the light that has passed the infrared transmission filter to capture an image of the puncture site, image processing means, which performs an extraction process of a vein from the captured image by the image capturing unit, a display unit, which displays the image processed by the image processor, and a power supply unit (not illustrated); the irradiating unit includes a plurality of light sources, which have optical axes inclined with respect to an optical axis of the image capturing unit at an angle A of 15° to 60°, and of a directional angle (2θ1/2) of the light irradiated from the light sources is 40° or more.

### TECHNICAL FIELD

This disclosure relates to a vein visualization device of a non-contact near-infrared system.

### BACKGROUND ART

Conventionally, in the field of medicine, when a person engaged in medical treatment injects a needle such as an injection needle or an intravenous feeding needle into an arm or the like of a patient, the person confirms a vein of a target to be tapped by visual check. However, it is sometimes difficult to confirm a position of the vein depending on the patient; therefore, a skill has been required for the person engaged in medical treatment. Accordingly, there has been proposed a device that irradiates a puncture site with near-infrared rays, photographs the reflected near-infrared rays with an infrared camera, and displays a vein part in a display unit of the device or the puncture site of a patient (for example, see Patent Literatures 1 to 5).
Patent Literature 1: JP-A-2013-22098
Patent Literature 2: JP-A-2011-160891
Patent Literature 3: JP-A-2011-212386
Patent Literature 4: JP-A-2006-102360
Patent Literature 5: JP-A-2004-267535

### SUMMARY

Like Patent Literature 1 or 2, in the case where the display unit of the device is a wearable computer such as a head-mounted display or a glasses type display, the person engaged in medical treatment is required to wear it whenever the person performs the tap work, leading to poor usability. Additionally, the wearable computer is often expensive. Like Patent Literature 3 or 4, the technique that projects a vein image on the puncture site of the patient requires advanced image processing and therefore the device is often expensive. Further, like Patent Literature 5, disposing an optical axis of a camera and an optical axis of a light source parallel causes a halation, making confirmation of the vein image difficult in some cases.

An object of this disclosure is to provide a compact, lightweight vein visualization device excellent in operability.

### Solution to Problem

A non-contact type vein visualization device according to the present invention includes an irradiating unit configured to irradiate a puncture site with light containing a wavelength component of 900 to 1500 nm; an image capturing unit that includes an infrared transmission filter, the image capturing unit being configured to receive the light that has passed the infrared transmission filter to capture an image of the puncture site; image processing means configured to perform an extraction process of a vein from the captured image by the image capturing unit; a display unit configured to display the image processed by the image processing means; and a power supply unit, wherein: the irradiating unit includes a plurality of light sources, the light sources having optical axes inclined with respect to an optical axis of the image capturing unit at an angle of 15° to 60°, and a directional angle 2θ1/2 of the lights irradiated from the light sources is 40° or more.

In the vein visualization device according to the present invention, it is preferable that a polarizing filter is not disposed on an optical path from the irradiating unit to the image capturing unit. While disposing the polarizing filter weakens the light received by the image capturing unit and therefore ISO sensitivity is required to be increased; and is likely to worsen clearness of the image, omitting the polarizing filter ensures obtaining a further fine image. Additionally, while disposing the polarizing filter fails to further reduce an aperture of a subject lens and therefore a depth of field is likely to shallow, omitting the polarizing filter allows preventing the shallow depth of field.

In the vein visualization device according to the present invention, it is preferable that a part of or all of respective irradiated regions of the light sources are superimposed in a visual filed range of the image capturing unit. The puncture site can be further uniformly illuminated, and consequently, the vein in the puncture site can be captured with more certainty.

In the vein visualization device according to the present invention, it is preferable that the irradiating unit is configured to emit pulsed light, the capturing timing of the image capturing unit is 10 to 30 images/second, and further comprising a control unit configured to synchronize a light emission timing of the irradiating unit with a capturing timing of the image capturing unit. Power consumption can be reduced.

In the vein visualization device according to the present invention, it is preferable that the irradiating unit is disposed at the first chassis, the display unit is disposed at the second chassis, the first chassis and the second chassis are coupled to be foldable, and the irradiating unit and the display unit are disposed at respective surfaces coming to outside when the first chassis and the second chassis are folded. A direction of the display unit can be adjusted to be an angle such that the worker easily sees the display unit, thereby improving working efficiency. Moreover, further downsizing can be achieved.

In the vein visualization device according to the present invention, it is preferable that the image capturing unit is disposed at the first chassis. This makes the additional downsizing possible; therefore, the vein visualization device is appropriate as a handy type.

In the vein visualization device according to the present invention, it is preferable that the image capturing unit is disposed at a third chassis fixed to the first chassis. Disposing the irradiating unit and the image capturing unit at the mutually different chassis allows appropriately providing a distance between the puncture site, and the irradiating unit and the image capturing unit.

The vein visualization device according to the present invention preferably further includes a supporting portion that vertically movably supports the third chassis. This configures the vein visualization device as a stand type; therefore, the worker can perform the tap work without holding the vein visualization device by hand. In view of this, the tap work is further simplified.

The vein visualization device according to the present invention preferably further includes a flexible arm. The worker can safely and reliably perform the tap work without holding the vein visualization device by the hand in a vehicle during traveling accompanied by vibrations, especially in an ambulance where many devices such as emergency treatment devices are loaded and therefore the work in a limited space is inevitable. In view of this, the tap work is further simplified.

### Advantageous Effects of Invention

This disclosure can provide a compact, lightweight vein visualization device excellent in operability.

### BRIEF DESCRIPTION OF DRAWINGS

FIG. 1 is a schematic front view illustrating a first example of a vein visualization device according to an embodiment.

FIG. 2 is one example of a characteristic diagram of emission of light from a light source used in the vein visualization device according to the embodiment.

FIG. 3 is a schematic diagram illustrating one example of a relationship between a visual filed range of an image capturing unit and respective irradiated regions from the light sources.

FIG. 4 is a schematic front view illustrating a second example of the vein visualization device according to the embodiment.

### DESCRIPTION OF EMBODIMENTS

While the following describes the present invention in detail showing embodiments, the present invention is not limitedly interpreted by these descriptions. As long as effects of the present invention are achieved, the embodiments may be variously modified.

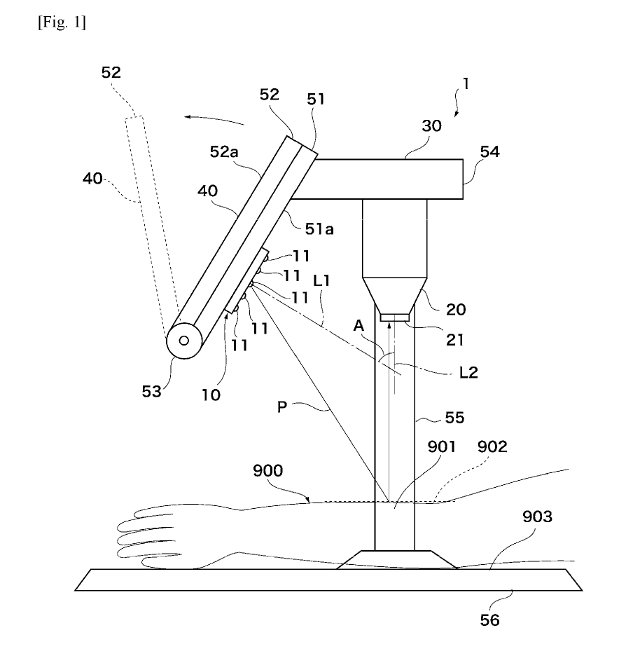

FIG. 1 is a schematic front view illustrating a first example of a vein visualization device according to the embodiment. A vein visualization device 1 according to the embodiment is a non-contact type vein visualization device comprising: an irradiating unit 10, which irradiates a puncture site 901 with light containing a wavelength component of 900 to 1500 nm, an image capturing unit 20, which includes an infrared transmission filter 21 and receives the light that has passed the infrared transmission filter 21 to capture an image of the puncture site 901, image processing means 30, which performs an extraction process of a vein from the captured image by the image capturing unit 20, a display unit 40, which displays the image processed by the image processing means 30, and a power supply unit (not illustrated); the irradiating unit 10 includes a plurality of light sources 11, which have optical axes L1 inclined with respect to an optical axis L2 of the image capturing unit 20 at an angle A of 15° to 60°, and of a directional angle 2θ1/2 of the light irradiated from the light sources 11 is 40° or more.

The irradiating unit 10 irradiates the puncture site 901 with lights containing a wavelength component of 900 to 1500 nm from the light sources 11. The light sources 11 are, for example, infrared LEDs. A peak wavelength of the light sources 11 is preferably 850 nm or 940 nm and more preferably 940 nm. It is only necessary that the irradiating unit 10 irradiates the light containing the wavelength component of at least 900 to 1500 nm, and, in addition to the wavelength component of 900 to 1500 nm, may irradiate the light containing the wavelength component of less than 900 nm and/or the wavelength component exceeding 1500 nm. Additionally, the irradiating unit 10 may include a visible light source (not illustrated) as necessary. The visible light source is the light source irradiating the light containing the wavelength component of 380 to 780 nm.

The puncture site 901 is, for example, a part of an arm portion 900 of a patient.

The image capturing unit 20 includes a lens and an imaging device. The lens condenses reflected light from the puncture site 901 and forms an image to a photo-receiving surface of the imaging device. The imaging device converts light and darkness of the light in the image formed by the lens into electrical signals. The imaging device is, for example, a CCD image sensor or a CMOS image sensor.

The image capturing unit 20, which includes the infrared transmission filter 21, does not include a heat-absorbing filter. The infrared transmission filter 21 is a filter that absorbs the visible light and transmits the infrared. The heat-absorbing filter is a filter that absorbs the infrared and transmits the visible light. Accordingly, since the image capturing unit 20 includes the infrared transmission filter 21 and does not include the heat-absorbing filter, an image of the reflected light in an infrared band can be captured.

The image processing means 30 inputs the electrical signals from the imaging device in the image capturing unit 20 to create the image displayed in the display unit 40. The image processing means 30 may adjust brightness or a contrast and the like of the image as necessary. Additionally, the image processing means 30 may perform a process to highlight a vein image, such as coloring the vein part in the image.

The display unit 40 displays the image processed by the image processing means 30. The display unit 40 is, for example, a liquid crystal panel. When the puncture site 901 is irradiated with the light containing the wavelength component of 900 to 1500 nm, the infrared is absorbed into the blood in the vein part; therefore, the reflectivity relatively lowers. Meanwhile, since the infrared is not absorbed into the blood but is reflected at tissues other than the vein, the reflectivity relatively heightens. Accordingly, the display unit 40 projects a vein pattern dark compared with other parts in the puncture site 901 and displays the image in which the vein is visualized. Furthermore, since the display unit 40 also projects a needle such as an injection needle or an intravenous feeding needle, a worker can perform the tap work while seeing the display unit 40 free from uncomfortable feeling. The present invention uses the light containing the wavelength component of 900 to 1500 nm to ensure obtaining the vein pattern with higher contrast by utilizing an increased absorbance of water compared with an absorbance of a deoxyhemoglobin in a wavelength band in which the wavelength is longer than 900 nm.

The power supply unit (not illustrated) may be a commercial power supply or a battery.

In the vein visualization device 1 according to the embodiment, it is preferable that a polarizing filter is not disposed on an optical path P from the irradiating unit 10 to the image capturing unit 20. The optical path P from the irradiating unit 10 to the image capturing unit 20 is a path of the light irradiated from the light sources 11 on the irradiating unit 10, reflected by the puncture site 901, and reaching the imaging device in the image capturing unit 20. Generally, while the use of the polarizing filter provides an effect of reducing a halation caused by regular reflection, an amount of transmitted light attenuates at the same time. The establishment of a system using a low-price, commercially available imaging device results in relatively low light sensitivity by CCD or C-MOS imagers near a near-infrared region (900 to 1000 nm); therefore, the attenuation of the amount of transmitted light by the polarizing filter deteriorates the image due to a noise. This embodiment adjusts an irradiation angle of the irradiating unit 10 to the optical axis L2 of the image capturing unit 20 to reduce the halation, rather than obtaining the deteriorated image due to the noise generated by sacrificing the amount of received light by the use of the polarizing filter, thus taking precedence of obtaining a clear image of less noise component consequently. While disposing the polarizing filter on the optical path P weakens the light received by the image capturing unit 20 and therefore ISO sensitivity is required to be increased; and is likely to worsen the clearness of the image, this embodiment does not include the polarizing filter to ensure obtaining a further fine image. Additionally, while disposing the polarizing filter fails to further reduce an aperture of the subject lens and therefore the depth of field is likely to shallow, this embodiment does not include the polarizing filter, thereby allowing preventing the shallow depth of field.

With this embodiment, the irradiating unit 10 includes the plurality of light sources (hereinafter sometimes referred to as first light sources) 11, which have the optical axes L1 inclined with respect to the optical axis L2 of the image capturing unit 20 at the angle A of 15° to 60°. The angle A formed by the respective optical axes L1 of the first light sources 11 and the optical axis L2 of the image capturing unit 20 is more preferable to be 30° or more. The halation can be prevented with more certainty even if the puncture site 901 has a curved surface. The angle A is further preferably 35° to 55°. The respective optical axes L1 of the first light sources 11 are straight lines extending in the traveling directions of the lights irradiated from the respective light sources 11, and the lights expand symmetrical with respect to these straight lines. FIG. 1 illustrates only the one optical axis L1 of the light sources 11 representing the optical axes L1 and omits the illustration of the optical axes of the light sources 11 other than this light sources 11. It is only necessary that the angle A formed by the respective optical axes L1 of the first light sources 11 and the optical axis L2 of the image capturing unit 20 is in a range of 15° to 60°, and there may be the optical axes parallel to one another or the optical axes facing in directions different from one another. The optical axis L2 of the image capturing unit 20 is a straight line passing through the center of the lens of the image capturing unit 20 and perpendicular to the surface of the lens. The direction of the optical axis L2 of the image capturing unit 20 is preferably a normal direction of a disposition-expected surface 902 for the puncture site. The disposition-expected surface 902 is an imaginary planar surface at a space at which the puncture site 901 is expected to be disposed and is a surface parallel to a work surface 903 on which the puncture site 901 is placed during the tap work. That is, in the case of performing the tap work by placing the puncture site 901 on a horizontal surface, the disposition-expected surface 902 is a horizontal surface. Additionally, in the case of performing the tap work by placing the puncture site 901 on a surface inclined with respect to the horizontal surface, the disposition-expected surface 902 is a surface inclined with respect to the horizontal surface according to the inclination of the surface on which the puncture site 901 is placed. The image capturing unit 20 captures the image of the puncture site 901 from right above, and thereby the worker easily grasps a sense of distance. The angle A formed by the respective optical axes L1 of the first light sources 11 and the optical axis L2 of the image capturing unit 20 of less than 15° likely to generate the halation, making the confirmation of the vein image difficult. The angle A formed by the respective optical axes L1 of the first light sources 11 and the optical axis L2 of the image capturing unit 20 exceeding 60° lowers the illuminance of the lights illuminating the puncture site, making the confirmation of the vein image difficult.

With this embodiment, the count of the first light sources 11 is preferably 2 to 30 pieces and more preferably 5 to 15 pieces. The count of the first light sources 11 of one piece narrows down the region that can be irradiated, failing to uniformly illuminate the puncture site 901. With this embodiment, configuring the count of the first light sources 11 plural ensures uniformly illuminating the puncture site 901. Consequently, the clearer vein images are obtainable.

With this embodiment, in addition to the first light sources 11, which have the optical axes L1 inclined at the angle A of 15° to 60° with respect to the optical axis L2 of the image capturing unit 20, the irradiating unit 10 may include a second light sources (not illustrated) having an optical axis inclined by less than 15° with respect to the optical axis L2 of the image capturing unit 20 and/or a third light sources (not illustrated) having an optical axis inclined at an angle exceeding 60° with respect to the optical axis L2 of the image capturing unit 20. A proportion of the count of the first light sources to the total count of the first light sources 11, the second light sources, and the third light sources is preferably 80% or more, more preferably 90% or more, and 100% is especially preferable.

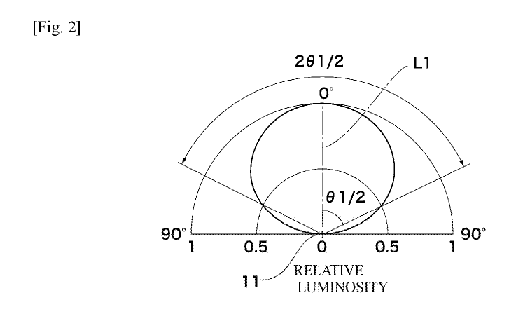

FIG. 2 is one example of a characteristic diagram of the emission of light from the light sources used in the vein visualization device according to the embodiment. The directional angle 2θ1/2 of the light irradiated from the first light sources 11 is 40° or more. The directional angle 2θ1/2 is more preferably 90° or more and further preferably 120° or more. The directional angle 2θ1/2 of less than 40° fails to uniformly illuminate the puncture site, thereby failing to obtain the clear vein image. Furthermore, to uniformly illuminate the puncture site, gaplessly disposing the considerably large number of light sources is necessary, resulting in a large device. A method for measuring the directional angle 2θ1/2 is to: fix the light sources 11 at the center of the circle, move a light receiving sensor along the circumference of the circle, measure the illuminance of the emitted light emitted from the light sources 11, normalize the illuminance on the optical axis L1 of the light sources 11 to define the maximum value of the illuminance as 1 (100%), and express a ratio of reduction in the illuminance when the optical axis L1 is inclined from the axis by θ with a diagram. Then an angle at which the illuminance becomes 0.5 (50%) is referred to as a half-value angle θ1/2 and a full angle found by summing both is referred to as a directional angle 2θ1/2.

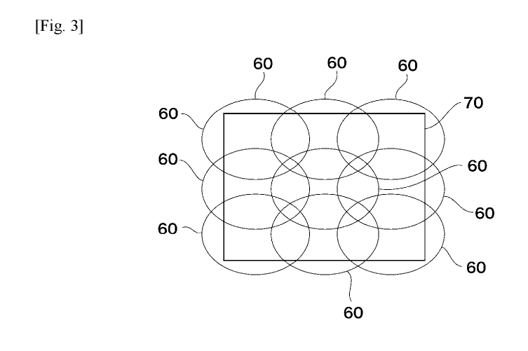

FIG. 3 is a schematic diagram illustrating one example of a relationship between a visual filed range of the image capturing unit and respective irradiated regions from the light sources. In the vein visualization device according to the embodiment, a part of or all of respective irradiated regions 60 of the light sources are preferably superimposed in a visual filed range 70 of the image capturing unit. The irradiated region 60 is a space irradiated by the lights irradiated from the first light sources 11 (illustrated in FIG. 1). The visual filed range 70 of the image capturing unit is a photographable space when the position of the image capturing unit 20 (illustrated in FIG. 1) is fixed for focalization at any distance and has a quadrangular pyramid shape having the optical axis L2 (illustrated in FIG. 1) of the image capturing unit as the central axis. FIG. 3 illustrates a cross-sectional surface of the irradiated regions 60 and the visual filedrange 70 perpendicular to the optical axis L2 of the image capturing unit at the photographing distance when the puncture site is focalized. As illustrated in FIG. 3, superimposing the irradiated regions 60 in the visual filed range 70 ensures uniformly illuminating the entire visual filed range 70. Then disposing the puncture site in this visual filed range 70 uniformly illuminates the puncture site. Consequently, the image of the vein can be captured across the entire puncture site with more certainty. The irradiated regions 60 are preferably superimposed at sites where the relative luminosity of the lights irradiated from the respective light sources 11 become 50 to 100%. This allows further narrowing down the aperture of the lens, ensuring deepening the depth of field.

With the vein visualization device 1 according to the embodiment (illustrated in FIG. 1), it is preferable that the irradiating unit 10 (illustrated in FIG. 1) emits pulsed light, a capturing timing of the image capturing unit 20 is 10 to 30 images/second, and a control unit (not illustrated) that synchronizes the light emission timing of the irradiating unit 10 with the capturing timing of the image capturing unit 20 (illustrated in FIG. 1) is further equipped with. Emitting the pulsed light ensures a reduction in power consumption. Furthermore, setting the capturing timing of the image capturing unit 20 to 10 to 30 images/second ensures obtaining a smooth moving image while reducing the cost and the power consumption. The capturing timing of the image capturing unit 20 is further preferable to be 15 to 25 images/second.

As illustrated in FIG. 1, with the vein visualization device 1 according to the embodiment, it is preferable that the irradiating unit 10 is disposed at a first chassis 51, the display unit 40 is disposed at a second chassis 52, the first chassis 51 and the second chassis 52 are coupled to be foldable, and the irradiating unit 10 and the display unit 40 are disposed at respective surfaces 51a and 52a, which come to outside when the first chassis 51 and the second chassis 52 are folded. Like the display unit 40 illustrated by the dotted line in FIG. 1, the direction of the display unit 40 can be adjusted to be an angle such that the worker easily sees the display unit 40, thereby improving working efficiency. Moreover, the device can be further downsized. The configuration of coupling the first chassis 51 and the second chassis 52 to be foldable is, for example, as illustrated in FIG. 1, the configuration of disposing a hinge 53 to couple the end of the first chassis 51 to the end of the second chassis 52.

As illustrated in FIG. 1, the vein visualization device 1 is preferably a stand type. Specifically, it is preferable that the vein visualization device 1 includes the first chassis 51 where the irradiating unit 10 is disposed, the second chassis 52 where the display unit 40 is disposed and which is coupled to the first chassis 51 to be foldable, a third chassis 54 fixed to the first chassis 51 and includes the image capturing unit 20 at the lower surface, and a supporting portion 55, which vertically movably supports the third chassis 54. Disposing the irradiating unit 10 and the image capturing unit 20 at the mutually different chassis 51 and 54 allows appropriately providing a distance between the puncture site 901, and the irradiating unit 10 and the image capturing unit 20 while providing the angle formed by the respective optical axes L1 of the light sources 11 and the optical axis L2 of the image capturing unit 20 at 15° to 60°, and additionally the device can be further downsized.

The first chassis 51 is preferably disposed to extend obliquely downward with respect to the third chassis 54. This ensures disposing the light sources 11 on the irradiating unit 10 closer to the puncture site 901 and ensures irradiating the light with higher illuminance to the puncture site 901. Consequently, the clearer vein images are obtainable.

The third chassis 54 may incorporate the image processing means 30.

As illustrated in FIG. 1, the lower end of the supporting portion 55 may fixed to a receiving table 56 on which the arm portion 900 of the patient is placed, or have a structure which is attachable to a workbench or the like by disposing a clip (not illustrated).

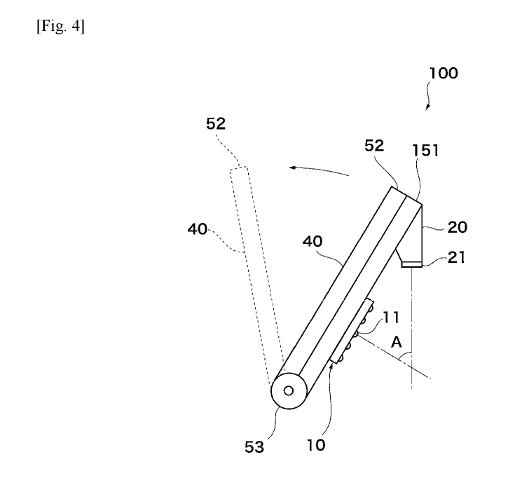

FIG. 4 is a schematic front view illustrating a second example of the vein visualization device according to the embodiment. In the vein visualization device 100 according to the embodiment, it is preferable that the image capturing unit 20 is disposed at the first chassis 151. The vein visualization device 100 of the second example illustrated in FIG. 4 is different from the vein visualization device 1 of the first example illustrated in FIG. 1 in that the image capturing unit 20 is disposed at the first chassis 151, and except for this configuration, the vein visualization device 100 has the basic configuration similar to that of the vein visualization device 1 of the first example. The identical reference numerals are assigned for the identical components between FIG. 1 and FIG. 4. The vein visualization device 100 illustrated in FIG. 4 allows additional downsizing. Additionally, since the vein visualization device 100 facilitates the works holding the vein visualization device 100 by the hand, the vein visualization device 100 is appropriate as a handy type. The image capturing unit 20 is preferably mounted to a surface on which the irradiating unit 10 is mounted in the first chassis 151.

As illustrated in FIG. 1 and FIG. 4, with the vein visualization devices 1 and 100 according to the embodiments, the irradiating unit 10, the image capturing unit 20, and the display unit 40 are configured as the integrated device, and thus having a lightweight, simple structure ensuring easily carrying the vein visualization devices 1 and 100. In view of this, regardless of indoor or outdoor and in any sort of traveling, for example, in a vehicle or in an airplane, the vein visualization devices 1 and 100 can be used, unnecessary to select the time and the location.

The vein visualization device 1 or 100 according to the embodiments may include a flexible arm (not illustrated). The flexible arm includes an arm portion, a first mounting portion disposed at one end of the arm portion to be mounted to the vein visualization device 1 or 100, and a second mounting portion disposed at the other end of the arm portion to be mounted to the receiving table 56, the workbench, or the like on which the arm portion 900 of the patient is placed. The arm portion is a rod-shaped or a tubular part employing a material or a structure that freely deforms and can hold the deformation state. The first mounting portion is, for example, a clip or a protrusion fitted to a mounting hole disposed at the vein visualization device 1 or 100. The first mounting portion may be removable to the vein visualization device 1 or 100 or may be integrated with the vein visualization device 1 or 100. The second mounting portion is, for example, a clip or a clamp. For example, in the vein visualization device 1 of the first example illustrated in FIG. 1, the supporting portion 55 may be replaced by the flexible arm. At this time, the first mounting portion of the flexible arm is preferably mounted to the first chassis 51, the second chassis 52, the hinge 53, or the third chassis 54. Additionally, with the vein visualization device 100 of the second example illustrated in FIG. 4, the first mounting portion of the flexible arm is preferably mounted to the first chassis 151, a second chassis 152, or the hinge 53.

By configuring the vein visualization devices 1 and 100 according to the embodiments as the stand type and by disposing the flexible arm, the worker can perform the tap work without the need for holding the vein visualization device 1 or 100 by the hand (hands-free). In view of this, the tap work is further simplified. Especially, disposing the flexible arm is appropriate for use in a vehicle during traveling accompanied by vibrations, especially in an ambulance where many devices such as emergency treatment devices are loaded and therefore the work in a limited space is inevitable. Furthermore, since the irradiating unit 10 and the image capturing unit 20 can be fixed to the patient at the appropriate position, obtaining the clearer vein images are possible.

### WORKING EXAMPLES

While the following gives explanations using the working examples of the present invention, the present invention is not limited to these examples.

### Working Example 1

The vein of the arm portion was observed using the vein visualization device 1 illustrated in FIG. 1. With the vein visualization device 1, 12 pieces of LEDs with the directional angle 2θ1/2 of 128° and the peak wavelength of 940 nm were used as the light sources 11. The plurality of light sources 11 were disposed such that the respective irradiation ranges were superimposed on the puncture site. The optical axes L1 were disposed such that the angle A formed by the respective optical axes L1 of the light sources 11 and the optical axis L2 of the image capturing unit fell in a range of 15° to 60°.

### Working Example 2

Working Example 2 was configured to similar to Working Example 1 except that the light sources 11 were replaced by LEDs with the directional angle 2θ1/2 of 44° and the peak wavelength of 940 nm.

### Comparative Example 1

Comparative Example 1 was configured to similar to Working Example 1 except that the light sources 11 were replaced by LEDs with the directional angle 2θ1/2 of 20° and the peak wavelength of 940 nm.

### Comparative Example 2

Comparative Example 2 was configured to similar to Working Example 1 except that the arrangement of the optical axes L1 was changed such that the angle A formed by the respective optical axes L1 of the light sources 11 and the optical axis L2 of the image capturing unit fell in a range of 0° to 10°.

### Comparative Example 3

Comparative Example 3 was configured to similar to Working Example 1 except that the arrangement of the optical axes L1 was changed such that the angle A formed by the respective optical axes L1 of the light sources 11 and the optical axis L2 of the image capturing unit fell in a range of 65° to 120°.

With Working Examples 1 and 2, irradiating the puncture site (the arm portion) with the lights from the light sources 11 both projected the vein pattern darker than the other parts in the puncture site 901 in the display unit 40 and displayed the clear vein image. Meanwhile, with Comparative Example 1, since the directional angle 2θ1/2 of the light sources 11 was too small, the puncture site was not able to be uniformly irradiated, resulting in a blurred vein image. With Comparative Example 2, since the angle A formed by the respective optical axes L1 of the light sources 11 and the optical axis L2 of the image capturing unit was too small, the halation occurred and the vein image was not able to be confirmed. With Comparative Example 3, since the angle A formed by the respective optical axes L1 of the light sources 11 and the optical axis L2 of the image capturing unit was too large, the illuminance of the light illuminating the puncture site became low, producing the blurred vein image.

### REFERENCE SIGNS LIST

1, 100 Vein visualization device
10 Irradiating unit
11 Light sources (first light sources)
20 Image capturing unit
21 Infrared transmission filter
30 Image processing means
40 Display unit
51, 151 First chassis
52 Second chassis
51a, 52a Surface coming to outside
53 Hinge
54 Third chassis
55 Supporting portion
56 Receiving table
60 Irradiated region
70 Visual filed range
900 Arm portion
901 Puncture site
902 Disposition-expected surface
903 Work surface
L1 Optical axis of light sources
L2 Optical axis of image capturing unit
P Optical path from irradiating unit to image capturing unit

What is claimed is:

1. A non-contact type vein visualization device comprising:
an irradiating unit configured to irradiate a puncture site with light containing a wavelength component of 900 to 1500 nm;
an image capturing unit that includes an infrared transmission filter, the image capturing unit being configured to receive the light that has passed the infrared transmission filter to capture an image of the puncture site;
image processing means configured to perform an extraction process of a vein from the captured image by the image capturing unit;
a display unit configured to display the image processed by the image processing means; and
a power supply unit, wherein:
the irradiating unit includes a plurality of light sources, the light sources having optical axes inclined with respect to an optical axis of the image capturing unit at an angle of 15° to 60°, and
a directional angle 2θ1/2 of the lights irradiated from the light sources is 40° or more.

2. The vein visualization device according to claim 1, wherein
a polarizing filter is not disposed on an optical path from the irradiating unit to the image capturing unit.

3. The vein visualization device according to claim 1, wherein
a part of or all of respective irradiated regions of the light sources are superimposed in a visual filed range of the image capturing unit.

4. The vein visualization device according to claim 1, wherein:
the irradiating unit is configured to emit pulsed light,
the capturing timing of the image capturing unit is 10 to 30 images/second, and further comprising a control unit configured to synchronize a light emission timing of the irradiating unit with a capturing timing of the image capturing unit.

5. The vein visualization device according to claim 1, wherein:
the irradiating unit is disposed at the first chassis,
the display unit is disposed at the second chassis,
the first chassis and the second chassis are coupled to be foldable, and
the irradiating unit and the display unit are disposed at respective surfaces coming to outside when the first chassis and the second chassis are folded.

6. The vein visualization device according to claim 5, wherein
the image capturing unit is disposed at the first chassis.

7. The vein visualization device according to claim 5, wherein
the image capturing unit is disposed at a third chassis fixed to the first chassis.

8. The vein visualization device according to claim 7, further comprising
a supporting portion that vertically movably supports the third chassis.

9. The vein visualization device according to claim 5, further comprising
a flexible arm.
